SEQUENCE LISTING (36 sequences)

SEQ ID NO: 1 - DNA, 20 bp, Homo sapiens
tgttaccaactgggacgaca

SEQ ID NO: 2 - DNA, 20 bp, Homo sapiens
ccatcacaatgcctgtggta

SEQ ID NO: 3 - DNA, 20 bp, Homo sapiens
gcagttgctagtcctacatc

SEQ ID NO: 4 - DNA, 19 bp, Homo sapiens
tcatccgcgtgaaacagcg

SEQ ID NO: 5 - DNA, 20 bp, Homo sapiens
gtatccgctgcctctacagc

SEQ ID NO: 6 - DNA, 20 bp, Homo sapiens
agcctggctcatcactgtct

SEQ ID NO: 7 - DNA, 23 bp, Homo sapiens
cttctcttggaaaaacgtgtggg

SEQ ID NO: 8 - DNA, 20 bp, Homo sapiens
tcatatcagcgtcctccatg

SEQ ID NO: 9 - DNA, 20 bp, Homo sapiens
ttgtcaatccggccatttgc

SEQ ID NO: 10 - DNA, 20 bp, Homo sapiens
ccgtatcttgggccacttgt

SEQ ID NO: 11 - DNA, 21 bp, Homo sapiens
caaagcacgctgcaaatacca

SEQ ID NO: 12 - DNA, 20 bp, Homo sapiens
ggccaaatgtgtcttccagt

SEQ ID NO: 13 - DNA, 23 bp, Homo sapiens
gcgcagcgatcggcgccggagat

SEQ ID NO: 14 - DNA, 30 bp, Homo sapiens
catacccggtggcaccctgttcttcagtca

SEQ ID NO: 15 - DNA, 41 bp, Homo sapiens
gctatttaggtgacactatagtcatagccaagttcccgttc

SEQ ID NO: 16 - DNA, 41 bp, Homo sapiens
ttgtaatacgactcactatagggccaggagttgttgaggaa

SEQ ID NO: 17 - DNA, 20 bp, Homo sapiens
agttgcatggcatgtgtgat

SEQ ID NO: 18 - DNA, 19 bp, Homo sapiens
catcactgggaacaccacg

SEQ ID NO: 19 - DNA, 20 bp, Homo sapiens
acctttccaagatggctcct

SEQ ID NO: 20 - DNA, 20 bp, Homo sapiens
cctgaaccacagacagagca

SEQ ID NO: 21 - DNA, 20 bp, Homo sapiens
ctcgcttgtcggccatgata

SEQ ID NO: 22 - DNA, 20 bp, Homo sapiens
cacgacttcttcaagtccgc

SEQ ID NO: 23 - DNA, 20 bp, Homo sapiens
aaccttgacttccgagcaga

SEQ ID NO: 24 - DNA, 20 bp, Homo sapiens
cttggggaacaaaggaatca

SEQ ID NO: 25 - DNA, 41 bp, Homo sapiens
gctatttaggtgacactatagatcatcacctttgccgagtc

SEQ ID NO: 26 - DNA, 43 bp, Homo sapiens
ttgtaatacgactcactatagggcttcttcccatctccatcca

SEQ ID NO: 27 - DNA, 23 bp, Homo sapiens
gagaacacatctacaactggtgg

SEQ ID NO: 28 - DNA, 20 bp, Homo sapiens
cctcgcttggaaaaccctct

SEQ ID NO: 29 - DNA, 20 bp, Homo sapiens
caggtgtttgagggtagctc

SEQ ID NO: 30 - DNA, 20 bp, Homo sapiens
cggttcatcatggtacagtc

SEQ ID NO: 31 - DNA, 20 bp, Homo sapiens
cacgagtggaaagcaactca

SEQ ID NO: 32 - DNA, 20 bp, Homo sapiens
ctgggaaaggtgtccctgta

SEQ ID NO: 33 - DNA, 39 bp, Homo sapiens
gctatttaggtgacactatagatggagggccatgtcaag

SEQ ID NO: 34 - DNA, 43 bp, Homo sapiens
ttgtaatacgactcactatagggaacaggctgccaataaaagc

SEQ ID NO: 35 - DNA, 19 bp, Homo sapiens
ccctgagattgggtcagcg

SEQ ID NO: 36 - DNA, 23 bp, Homo sapiens
agagcagttattcacctcaaacc

(19) United States
(12) Patent Application Publication
(10) Pub. No.: US 2022/0315889 A1
(43) Pub. Date: Oct. 6, 2022

(54) MATERIALS AND METHODS FOR GENERATING FUNCTIONAL OOCYTES

(71) Applicant: The Brigham and Women's Hospital, Inc., Boston, MA (US)

(72) Inventor: Raymond Manohar ANCHAN, Boston, MA (US)

(21) Appl. No.: 17/619,930

(22) PCT Filed: Jun. 17, 2020
(86) PCT No.: PCT/US2020/038139

§ 371 Date: Dec. 16, 2021

Related U.S. Application Data
(60) Provisional application No. 62/862,333, filed on Jun. 17, 2019.

(51) Int. Cl. C12N 5/075; B01L 3/00

(52) U.S. Cl. CPC C12N 5/0609; B01L 3/50; C12N 2501/31; C12N 2501/392; C12N 2501/905; C12N 2506/45

(57) ABSTRACT

The invention relates to devices, compositions, and treatment of infertility inter alia by generating not only functional ovarian tissue but also functional oocytes from induced pluripotent stem cells. Treatments for hormone replacement are also described. In one aspect, the invention features a container including (i) a substrate; (ii) human iPSCs capable of differentiating into functional ovarian tissue; and (ii) a culture media.

### CROSS-REFERENCE TO RELATED APPLICATIONS

This application claims benefit of U.S. Provisional Application No. 62/862,333, filed Jun. 17, 2019, the contents of which are incorporated herein by reference in their entirety.

### SEQUENCE LISTING

The instant application contains a Sequence Listing which has been submitted electronically in ASCII format and is hereby incorporated by reference in its entirety. The ASCII copy, created on Jun. 12, 2020, is named 51420-002WO2_Sequence_Listing_06.12.20_ST25 and is 5,592 bytes in size.

### BACKGROUND OF THE INVENTION

This invention relates to fertility and endocrine function in females.

Despite significant and rapid advances in assisted reproductive technologies (ART), few fertility-preserving options remain for women of reproductive age who have cancer and are planning chemotherapy (Hyman et al., Clin. Med. Insights Reprod. Health 7, 61-69 (2013)). Many chemotherapeutic regimens are gonadotoxic, leaving women with compromised fertility after treatment. Currently, the most reliable method for fertility preservation is pre-chemotherapy fertility treatment cycles using ART to induce the growth of multiple oocytes for cryopreservation either as oocytes or after fertilization as embryos (Blumenfeld et al., Curr. Opin. Obstet. Gynecol. 15, 359-370 (2003); Srikanthan et al., Mol. Clin. Oncol. 8, 153-158 (2018)). Unfortunately, referral rates for ART prior to commencing gonadotoxic chemotherapy are low (Bastings et al., Hum. Reprod. 29, 2228-2237 (2014)). Moreover, compared to similar women without cancer, even women with apparently preserved ovarian function after chemotherapy are likely to experience diminished ovarian reserve with less success from ART cycles with autologous oocytes (Luke et al., Hum. Reprod. 31, 183-189 (2016)). This is compounded in this patient population by the fact that some mutations which predispose women to cancer, such as BRCA1 and Fanconi Anemia pathway members, are also associated with premature ovarian insufficiency (Sklavos et al., J. Clin. Endocrinol. Metab. 99, 1608-1614(2014); Daum et al., Fertil. Steril. 109, 33-38 (2018)).

Beyond infertility, premature ovarian insufficiency following chemotherapy treatment causes additional sequelae of estrogen deficiency (Rose et al., Nat. Rev. Endocrinol. 12, 319 (2016)). Cancer survivors experience accelerated bone loss, increased sexual dysfunction, and higher rates of cardiovascular mortality compared to their age-matched peers without cancer (Stava et al., J. Cancer Surviv. 3, 75-88 (2009); Podfigurna-Stopa et al., J. Endocrinol. Invest. 39, 983-990 (2016); Lindau et al., Am. J. Obstet. Gynecol. 213, 166-174(2015); Boyne et al., Cancer Med. 7, 4801-4813 (2018)). Restoration of hormones is primarily accomplished by synthetic hormone replacement therapy (HRT); however, there is limited data on the long-term health consequences of prolonged HRT in adolescents and young women, raising concerns about increasing risks of secondary malignancies (Fish et al., J. Pediatr. Adolesc. Gynecol. 24, 98-101 (2011); Sullivan et al., Fertil. Steril. 106, 1588-1599 (2016); Shanis et al., Semin. Hematol. 49, 83-93 (2012)).

Against this background, there is a need in the art for methods and compositions to preserve or restore ovarian function.

### SUMMARY OF THE INVENTION

In one aspect, the invention features a container including (i) a substrate; (ii) human iPSCs capable of differentiating into functional ovarian tissue; and (ii) a culture media. In some embodiments, the container is configured such that the culture media continuously flows through the container. In another embodiment, the container has an inlet port and an outlet port configured such that media flows through the container. In other embodiments, the iPSCs are matched to an individual female. In still other embodiments, the substrate is a microfluidic chip. In yet other embodiments, the container includes human embryoid bodies (EB), human steroidogenic cells, human ovarian tissue, human oocytes, or, human reproductive hormones (for example, the hormones are progesterone, estradiol, testosterone, or anti-Müllerian hormone (AMH) or a combination thereof). In other embodiments, the culture medium includes human follicular fluid (HFF).

In other aspects, the invention features ovarian tissue produced by the steps of i) providing human iPSCs on a substrate; ii) differentiating the iPSCs into ovarian tissue; and iii) harvesting the ovarian tissue. In some embodiments, the substrate is a microfluidic chip. In other embodiments, the iPSCs are autologous.

In yet other aspects, the invention features human oocytes produced by the steps of i) providing human iPSCs on a substrate; ii) differentiating the iPSCs into oocytes; and iii) harvesting the oocytes. In some embodiments, the substrate is a microfluidic chip. In other embodiments, the iPSCs are autologous.

In still other aspects, the invention features conditioned media produced by the steps of i) providing human iPSCs on a substrate; ii) providing a cell culture medium which contacts and continuously flows over the iPSCs; iii) differentiating the iPSCs into ovarian tissue or oocytes; and iv) collecting, following the contacting, the media, thereby producing conditioned media. In some embodiments, the substrate is a microfluidic chip. In other embodiments, the iPSCs are autologous. In still other embodiments, the conditioned cell culture medium includes HFF. In yet other embodiments, the collected conditioned media includes reproductive hormones (for example, the reproductive hormones are progesterone, estradiol, testosterone, or AMH or a combination thereof).

In still other aspects, the invention features methods of using the aforementioned ovarian tissue, oocytes, or conditioned culture media for treating fertility or endocrine conditions in a human female.

Accordingly, in yet another aspect, the invention features treating a fertility or endocrine condition in a human female, the method including administering the aforementioned produced human ovarian tissue produced to the female. In some embodiments, administering includes implanting the ovarian tissue into the female. In still other embodiments, the method includes administering conditioned media. Exemplary fertility or endocrine conditions to be treated include premature menopause, premature ovarian insufficiency, infertility, chemotherapy-induced premature ovarian failure, chemotherapy-induced diminished ovarian reserve, chemotherapy-induced decreased ovarian reserve, idiopathic premature ovarian failure, chemotherapy-induced ovarian failure, chemotherapy-induced premature ovarian insufficiency, estrogen deficiency, age-related rapid follicular atresia, follicular atrophy, post-chemotherapy ovarian insufficiency, and oophorectomy.

In another aspect, the inventions features treating a fertility or endocrine condition in a human female, the method including administering the aforementioned produced human oocytes to the female. In some embodiments, administering includes implanting the oocytes into the female. In still other embodiments, the method includes administering conditioned media. Exemplary fertility or endocrine conditions to be treated include is premature ovarian insufficiency, infertility, chemotherapy-induced premature ovarian failure, chemotherapy-induced diminished ovarian reserve, chemotherapy-induced decreased ovarian reserve, idiopathic premature ovarian failure, chemotherapy-induced ovarian failure, chemotherapy-induced premature ovarian insufficiency, post-chemotherapy ovarian insufficiency, and oophorectomy.

In still another aspect, the invention features a method of providing a hormone replacement therapy to a human female, the method including administering the aforementioned produced conditioned media to the female. In some embodiments, the conditioned media includes human reproductive hormones. Exemplary reproductive hormones include progesterone, estradiol, testosterone, or AMH or a combination thereof.

The compositions and methods provide several useful clinical benefits. Indeed, the invention provides for an autologous iPSC system for the de novo generation of functional oocytes. Moreover, oocyte generation from iPSCs is enhanced by using human follicular fluid as is described herein. The invention also provides for using stem cell-derived ovarian cortex to support increased generation of differentiating stem cells for establishing primitive oogonia. For example, using the compositions and methods described herein, ovarian hormonal function and fertility may be preserved or restored in several patient populations, including (1) the 8% of childhood cancer survivors who will undergo premature menopause by age 40, (2) the nearly 30% of breast cancer cases that are women younger than 50 years old, (3) women with chemotherapy-induced premature ovarian failure or diminished ovarian reserve, (4) women with irregular menstrual cycles and estrogen deficiencies; and/or (5) women with idiopathic premature ovarian failure. As is described in further detail in the Examples below, the iPSC-generated ovarian tissue preserves or restores fertility by promoting neo-gametogenesis of functional stem cell-derived germ cells, and is useful for treating premature menopause by restoring or preserving ovarian hormonal function. Additionally, the iPSC-generated ovarian tissue is chemoprotective against loss of ovarian hormone function, as indicated by the physiologic levels of hormones produced such as estrogen, estradiol, and progesterone. The ability to generate patient specific bioidentical hormones as well as potentially autologous gametes further provides useful therapeutic options for the aforementioned women. In these ways, and in others described herein, the devices, compositions and methods described herein achieve significant and advantageous effects.

Other features and advantages of the invention will be apparent from the following description of the preferred embodiments thereof, and from the claims.

### BRIEF DESCRIPTION OF THE DRAWINGS

FIG. 1 shows that mGriPSCs differentiate into steroidogenic cells that express ovarian and oocyte markers. In situ hematoxylin & eosin (H&E) staining of mouse ovarian sections shows follicles (I. A-C). Immunohistochemistry (IHC) demonstrates mouse ovarian localization of the known ovarian antigens FSHR, INHB, FOXL2, CYP19A1, AMHR2 (I. D-I) and oocyte markers ZP1, ZP2, DAZL, DDX4 (I. J-O). In vitro phase-contrast images of a mGriPSC colony (II. A), mGriPSC embryoid bodies (EBs) at two weeks post-suspension (II. B), and an attached mGriPSC EB (II. C). Immunocytochemistry (ICC) shows that differentiated mGriPSC EBs at 15 days post-attachment also express the same corresponding ovarian (II. D-H) and oocyte antigens (II. I-L). Transcriptome analysis using RT-PCR confirms synthesis of these as well as other related ovarian tissue transcripts in these mGriPSCs EBs (Amhr2, Cyp19a1, Fshr, Gja1, Amh, Ddx4, Boule, Blimp1; III). These differentiated EBs also synthesize the reproductive ovarian hormone, estradiol (E2), at physiologically relevant levels over 15 days (710-1220 pg/mL; IV). E2 synthesis is further enhanced by the addition of human follicular fluid (HFF) to culture media (0%, 1%, and 5% HFF; n=3, Mann-Whitney U test, P<0.05; V). HFF also promotes increased expression of germ cell marker ZP1 (zona pellucida 1) and a granulosa cell marker GJA121 (gap junction protein alpha 1; n=7, Mann-Whitney U test, P<0.003; VI). Data represented as mean±SEM. Arrows on images indicate areas of positive immunostaining.

FIG. 2 shows fluorescence activated cell sorting (FACS) purifies functional iPSC-derived steroidogenic ovarian cells. Schematic representation of the experimental flow for generation of iPSCs, green fluorescent protein (GFP) labeling, differentiation, and FACS purification of AMHR2+ mGriPSCs-GFP (A). FACS revealed 0.7% of the total population of mGriPSCs-GFP positively expresses ovarian antigen AMHR2 (P4 population; B). Pre- and post-sorted mGriPSCs-GFP synthesize E2 and progesterone (P4) through 15 days in culture (C, D), while post-sorted cells primarily produce E2, consistent with our prior observations17 (E). ICC showed that differentiated mGriPSCs-GFP both before (F-H) and after (I-K) FACS express the ovarian antigens AMHR2, CYP19A1, and GJA1. The expression of these ovarian markers is further supported by RT-PCR transcriptome analysis of pre- (L) and post-sorted (M) cells (Amhr2, Cyp19a1, Fshr, Gja1, Amh, Ddx4, Boule, and Blimp1). Data represented as mean±SEM. Arrows on images indicate areas of positive immunostaining.

FIG. 3 shows AMHR2+ sorted mGriPSCs-GFP preserve estrogen effects but do not form teratomas. Schematic representation of in vivo experimental flow for ovarian injections (A). Following alkylating chemotherapy, mice injected intramuscularly with sorted or unsorted mGriPSCs show evidence of mammary pad regeneration after 72 hours, whereas mice that did not receive mGriPSCs show no mammary pad recovery (B). H&E staining revealed partial ovarian atrophy after chemotherapy exposure, as indicated by a decrease in ovary size and fewer number of follicles (C), as compared to a control ovary (D). Injecting FACS-purified cells prevents the formation of teratomas, as compared with unsorted cell injections (E, F; data represented as mean±SEM). Compared to mice that receive chemotherapy without mGriPSCs injections, mice that receive sorted or unsorted mGriPSCs injections show an increase in blood E2 levels after treatment (G; n=3, data represented as a percent change in hormone concentrations from pre-chemotherapy to post-chemotherapy/mGriPSCs injections, as measured by ELISA). As previously reported, since E2 is the predominant reproductive hormone synthesized by differentiating mGriPSCs, P4 is observed to remain unchanged under all conditions17 (G). Testosterone is only detectable in mice that receive chemotherapy but no mGriPSCs injections (G; also see FIG. 7).

FIG. 4 shows neo-gametogenesis with differentiated iPSCs in vivo. Injection of sorted GFP-labeled mGriPSCs into mouse ovaries following chemotherapy results in de novo generation of oocytes (A). Reverse transcription quantitative PCR (RT-qPCR) analysis shows injection of sorted mGriPSCs promotes in vivo expression of Zp1 (B; n=3, Mann-Whitney U test, p<0.05). Images C-O depict cells and tissues derived from AMHR2+ mGriPSC-GFP injected mouse ovaries that were exposed to chemotherapy. Phase contrast image of mature oocytes with their surrounding granulosa cells (C). Fluorescent microscopy revealed that the collected mature oocytes express GFP (D, E). IHC on mGriPSCs-GFP injected ovaries indicated localization of GFP to granulosa cells of the follicle (F). IHC also showed co-localization of AMHR2 (G) and DAZL (H) with GFP in the ovarian follicles of treated mice. Isolated granulosa cells also expressed GFP (I). Collectively, these results demonstrate that GFP-labeled mGriPSCs contribute to follicular formation. Fluorescent microscopy displays calcium ionophore (A23817) activation of mature oocytes (J, K). Furthermore, maturation and functionality of GFP-labeled oocytes was supported by the ability to fertilize some of these oocytes in vitro (L-P). Data represented as mean±SEM. Arrows on images indicate areas of positive immunostaining.

FIG. 5 shows sorted mGriPSCs promotes gametogenesis and synthesizes AMH in vitro and in vivo. ICC demonstrates co-localization of oocyte markers DDX4 and ZP1 in differentiating mGriPSCs under various co-culture conditions (A-C, stem cell colonies; D-F, suspended EBs; G-I, attached EBs). Differentiating SCs in the three aforementioned states were co-cultured in a transwell with either no feeder layer, irradiated mouse embryonic fibroblasts (MEFS), G4 mouse ESCs, unsorted mGriPSCs EBs, or AMHR2+ sorted mGriPSCs (FIG. 9). FACS sorting demonstrated a higher percentage of cells express the oocyte markers, BOULE and ZP2, in suspended EBs co-cultured with AMHR2+ sorted cells compared with EBs co-cultured in other conditions (J, K). RT-PCR of the AMHR2+ sorted feeder layer shows expression of ovarian markers (L). RT-qPCR demonstrated greater expression of oocyte markers, ZP1 and DDX4, in suspended EBs co-cultured with sorted cells compared with EBs co-cultured with unsorted cells (M). These cells also continued to synthesize estradiol under all co-culture conditions (N). Of interest, differentiating mGriPSCs produced AMH both in vitro co-culture (O) and in vivo (R, S; n=3, Mann-Whitney U test, p<0.05, ±SE). IHC of experimental ovaries confirmed estradiol and AMH synthesis following injection (P, Q).

FIG. 6 shows an autologous stem cell-based model for fertility and endocrine function restoration. A model for translational applications is as follows. Somatic cells are collected from a patient (A) and converted into autologous iPSCs (B). These iPSCs are differentiated into ovarian steroidogenic and reproductive cells (C, D). Microfluidic chip technology may be utilized to create a sterile, continuously flowing environment, creating the ability to purify autologous bioidentical reproductive hormones from the conditioned media (D). The autologous oocytes (E) and bioidentical steroid hormones (D) may then be collected and used to treat the same patients, ultimately restoring fertility and endocrine function (F).

FIG. 7 shows that chemotherapeutic agents decreased estradiol production, while injection of unsorted stem cells resulted in teratoma formation. Busulfan and Cyclophosphamide injections result in breast atrophy in nude mice compared to control mice not receiving any injection. Restoration of breast tissue was observed after subsequent injection of unsorted differentiated mGriPSCs (A). Estradiol synthesis decreased in mice that received Busulfan and Cyclophosphamide compared to control mice. Estradiol production was measured by ELISA over 4 timepoints (B). Teratoma formation developed from injections of unsorted differentiated mGriPSCs. Ovarian teratomas from intraovarian injections inhibited oocyte retrieval (C). Intramuscular injections of unsorted differentiated mGriPSCs in the hind leg led to teratoma formation (D1). The injection of AMHR2+ sorted differentiated mGriPSCs in the hind leg did not demonstrate any teratoma formation (D2).

FIG. 8 shows oocytes collected from the ovaries of nude mice expressed GFP and were parthenogenically activated or fertilized. Nude mice received direct intraovarian injections of GFP labeled AMHR2+ mGriPSCs following injection of busulfan and cyclophosphamide. 8 total oocytes fluoresced green (A and B) and subsequent activation by fertilization (C-H) or parthenogenesis (I-L) was attempted.

FIG. 9 shows a schematic representation of co-culture experimental setup. The bottom layer of the transwells were plated with either attached mGriPSC-GFP EBs (a) or FAC sorted AMHR2+ mGriPSCs (b). The top layer of the transwells were plated with mGriP stem cells (1), the suspended mGriPSC EB (2), or the attached mGriPSC EB (3). Media was collected every 3 days for 15 days from each of the 6 different conditions to analyze hormone production via ELISA at different time points of cell culture. At day 15, further analysis was performed via immunocytochemistry, FACS, and RNA extraction for RT- and QT-PCR.

FIG. 10 shows FACS indicated positive expression of ZP2 (405), GDF9 (594), and DDX4 (647). Positive gating was consistent through all experimental conditions, as it was referenced to the unstained control. Positive cell populations exist in each experimental condition.

FIG. 11 shows FACS indicated positive expression of ZP2 (405), BOULE (594), and DDX4 (647). Positive gating was consistent through all experimental conditions, as it was referenced to the unstained control. Positive cell populations exist in each experimental condition.

### DETAILED DESCRIPTION OF THE INVENTION

In this description useful devices, compositions, and treatment of infertility inter alia by generating functional oocytes derived from induced pluripotent stem cells are described. Treatments for hormone replacement are also described.

Many oncologic therapies given to young women are gonadotoxic and associated with diminished ovarian reserve, risk of permanent sterility, and premature menopause. Derivation of steroidogenic ovarian cells from induced pluripotent and embryonic stem cells are available. Derived cells not only produced reproductive hormones, but also displayed markers of ovarian tissue and primordial gametes. Below we describe that human follicular fluid, when added to a stem cell differentiation system, enhances the steroidogenic potential of derived cells and increases the subpopulation of cells that differentiate to express the ovarian and germ cell markers GJA1 and ZP1, respectively. Using an in vivo model of chemotherapy-induced premature ovarian insufficiency in nude mice, it is demonstrated that orthotopic implantation of these derived cells restored ovarian hormonal production and produces functional stem-cell derived germ cells. Collectively, these data show that stem cell derived steroidogenic ovarian tissue is useful to promote neo-gametogenesis and treat premature menopause.

Indeed, by using patient-specific iPSCs to generate steroidogenic ovarian tissue, these derived cells can be syngeneic with the patient. Below we demonstrate that differentiation of mouse iPSCs into steroidogenic reproductive ovarian tissue in vitro is enhanced by media containing human follicular fluid. Following differentiation, we demonstrate that these ovarian and primordial oocytes can be isolated through fluorescence-activated cell sorting (FACS) using a cell surface receptor for a biochemical marker of ovarian reserve, anti-Mullerian hormone receptor 2 (AMHR2). Injection of the stem cell derived-AMHR2+ cells into subfertile mice exposed to gonadotoxic chemotherapy restored ovarian function as indicated by both the recovery of steroidogenic production and de novo generation of stem-cell derived gametes. Finally, these stem cells are shown to trigger activation of the endogenous oogenesis process to produce functionally mature oocytes as demonstrated by their capacity for fertilization and activation via a calcium ionophore towards the generation of parthenotes (Choi et al., J. Tissue Eng. Regen. Med. 12, e142-e149 (2018)).

The following abbreviations found in Tables 1a and 1b are used in this application.

TABLE 1a
Abbreviations List

| Name | Abbreviation |
|---|---|
| 2-mercaptoethanol | BME |
| 4′,6-diamidino-2-phenylindole | DAPI |
| anti-Müllerian hormone | AMH |
| anti-Müllerian hormone receptor 2 | AMHR2 |
| Advanced reproductive therapy | ART |
| Aromatase | CYP19 |
| Chemotherapy-related amenorrhea | CRA |
| DEAD-box helicase 4 | DDX4 |
| Deleted in zoospermia-like | DAZL |
| Dulbecco's Modified Eagle Medium/ | DMEM-F12 |
| Nutrient F-12 | |
| Embryonic stem cell research oversight- | ESCRO-LIF |
| leukemia inhibitory factor | |
| Embryonic stem cells | ESCs |
| Enzyme-linked immunosorbent assay | ELISA |
| Fluorescent activated cell sorting | FACS |
| Follicle-stimulating hormone | FSHR |
| Forkhead box protein L2 | FOXL2 |
| Gap junction alpha-1 | GJA1 |
| Gonadotropin releasing hormone | GnRH |
| Granulosa cell | GC |
| Green fluorescent protein | GFP |
| Heat-inactivated fetal bovine serum | HI FBS |
| Hematoxylin and eosin | H&E |

TABLE 1b
Abbreviations List

| | |
|---|---|
| Hormone replacement therapy | HRT |
| Human chorionic gonadotropin | hCG |
| Human follicular fluid | HFF |
| Immunocytochemistry | ICC |
| In vitro fertilization | IVF |
| Induced pluripotent stem cells | iPSCs |
| Inhibin β-A | INHIB |
| Institution of Animal Care and Use | IUCAC |
| Committee | |
| Knock-out serum replacement | KSOR |
| Mouse embryonic fibroblasts | MEFs |
| Mouse granulosa cell derived-induced | mGriPSCs |
| pluripotent stem cells | |
| Non-essential amino acids | NEAA |
| Optimizing cutting temperature | OCT |
| Phosphate-buffered saline | PBS |
| Polymerase chain reaction | PCR |
| Potassium simplex optimized medium | KSOM |
| PR domain zinc finger protein-1 | BLIMP-1 |
| Pregnant mare serum gonadotropin | PMSG |
| Premature ovarian failure | POF |
| Premature ovarian insufficiency | POI |
| Stage-specific embryonic antigen-1 | SSEA-1 |
| Virus-G glycoprotein | VSV-G |
| Zona pellucida | ZP |

### Results

Differentiated iPSCs Regenerate Steroidogenic Reproductive Ovarian Tissue In Vitro.

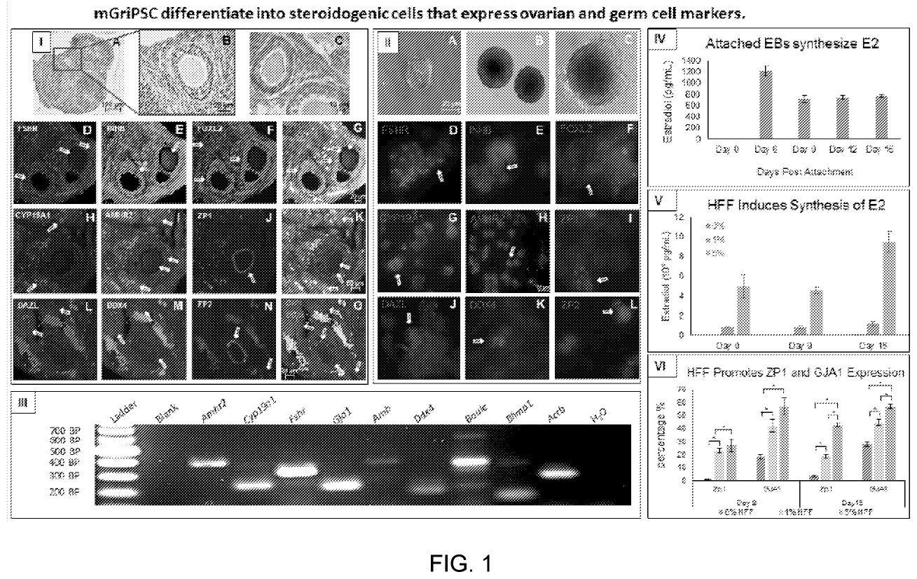

Age-matched normal mouse ovarian tissue (FIG. 1:I A-C) and in vitro differentiated mGriPSCs at 2 weeks post-attachment (FIG. 1:II A-C) expressed comparable antigen profiles (FIG. 1:I D-O and FIG. 1:II D-L) for the ovarian and germ cell markers AMHR2, CYP19A1, FOXL2, FSHR, INHB, DAZL, DDX4, ZP1, and ZP2 using immunofluorescent staining. Expression of these markers in the differentiated mGriPSCs was also confirmed by RT-PCR (FIG. 1:III). Steroidogenic activity in embryoid bodies (EBs) from the differentiated mGriPSCs was confirmed by ELISA measurement of physiological concentrations of estradiol in culture media during extended cell culture (FIG. 1IV).

Human Follicular Fluid Promotes Efficient Differentiation of iPSCs into Steroidogenic Ovarian Tissue and Enhances Expression of ZP1 and GJA1.

mGriPSCs were cultured with media containing 1% or 5% human follicular fluid (HFF) obtained at the time of oocyte retrieval. The addition of HFF markedly increased the estradiol synthesis by mGriPSCs over 15 days in culture (FIG. 1V). Additionally, HFF promoted increased expansion of a subpopulation of cells expressing the granulosa cell marker GJA1 (Su et al., Semin. Reprod. Med. 27, 32-42 (2009)) and the oocyte marker ZP1 (FIG. 1VI).

Stem Cell Derived Steroidogenic Ovarian Tissue Retains Endocrine Function when Isolated Using Fluorescence-Activated Cell Sorting (FACS).

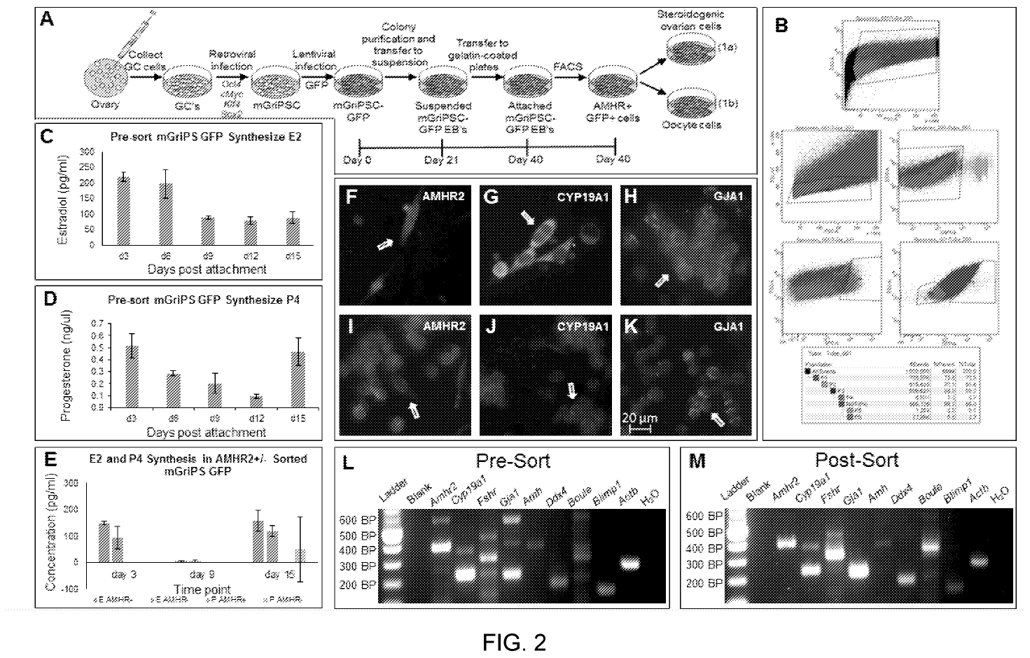

After derivation in vitro, mGriPSCs were stably transfected with a green fluorescent protein (GFP) reporter using a lentivirus and then differentiated into ovarian tissue as described (Anchan et al., PLOS ONE 10, e0119275 (2015); Lipskind et al., Reprod. Sci. 25, 712-726 (2017))(FIG. 2A). These cells were then sorted by FACS using a cell surface level antigen, AMHR2+, specific to differentiated ovarian tissue (FIG. 2B). Functional endocrine analysis revealed preservation of estradiol and progesterone synthesis capacity in both pre- and post-sorted cells (FIG. 2C-E). ICC indicated retained expression of ovarian antigens AMHR2+, CYP19A1, and GJA1 in pre-sort (FIG. 2F-H) and post-sort cells (FIG. 21-K). RT-PCR further confirmed these results, as indicated by the retention of RNA transcript expression of ovarian and oocyte-specific genes (Amhr2, Cyp19a1, Fshr, Gja1, Amh, Ddx4, Boule, and Blimp1; FIG. 2L, M). Sorting mGriPSCs with AMHR2 thus leads to a heterogenous population of differentiated cells that comprise of both putative granulosa cells and oocytes,

### Alkylating Chemotherapy Leads to Accelerated Follicular Atresia and Breast Atrophy in Nude Mice.

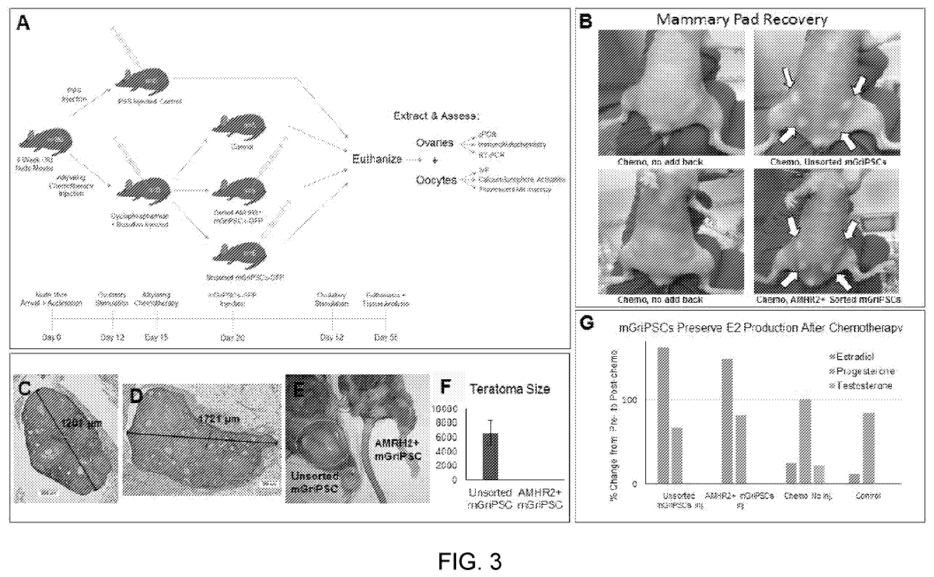

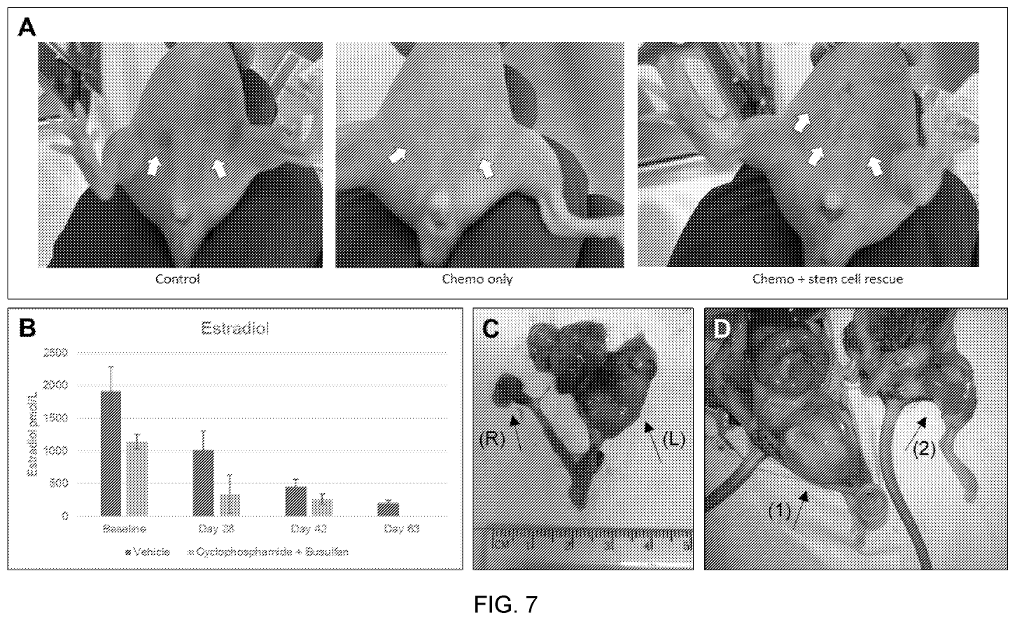

Nude mice are subfertile from premature ovarian insufficiency due to age-related rapid follicular atresia (Rebar et al., Endocrinology 108, 120-126 (1981)). To model the additive impact of alkylating chemotherapy to a subfertile population, nude mice received single intraperitoneal injections of busulfan (12 mg/kg) and cyclophosphamide (120 mg/kg) or 100 ul of vehicle (10% DMSO in PBS; FIG. 3A). Within 63 days of chemotherapy injections, mice displayed smaller ovaries with fewer follicles (FIG. 3C) compared to age-matched controls (FIG. 3D), in addition to breast atrophy (FIG. 7A) and a complete loss of estradiol production (FIG. 7B). This accords with previous mouse studies that showed the gonadotoxic effects of cyclophosphamide and busulfan (Jiang et al., J. Zhejiang Univ. Sci. B 14, 318-324 (2013)).

The gonadotoxicity of chemotherapeutic agents is highly variable and dependent on dose and patient age. Dosing related toxicity is influenced by absolute dosing and cumulative dosing. Nearly 30% of breast cancer cases present in women younger than 50 years old. While newer treatment regimens employed are less gonadotoxic, regimens still consist of combination medications that include cyclophosphamide, known to deplete the number of primordial follicles, thereby potentially leading to infertility. For common regimens such as adriamycin/cytoxan (AC), the risk of premature ovarian failure was thought to be largely dependent on patient age, with the risk of complete ovarian failure <10% in women <30, and nearly 100% in women >40; however other studies indicate that AC is considered to have intermediate risk for gonadotoxicity in women >40 years age. As such, these results demonstrate decreased ovarian follicles, marginal atrophy and presumably an increased risk for ovarian failure and infertility after the chemotherapeutic gonadotoxic insult. Therefore, this mouse model with chemically depleted ovarian follicles resembles the clinical paradigm encountered by reproductive age women undergoing chemotherapy by showing decrease but not total elimination of follicles.

FACS-Sorted AMHR2+ mGriPSCs Restore Hormonal Function but do not Form Teratomas.

mGriPSCs are known to produce estradiol, but like all iPSCs, these form teratomas when injected into mice (Anchan et al., Curr. Protoc. Hum. Genet. (2017))(FIG. 3E FIG. 7C). To confirm that sorting mGriPSCs based on the expression of cell surface protein AMHR2 might allow selection of a more differentiated cell type, thereby minimizing the risk of tumor formation the following experiment was conducted. Six-week old nude female mice underwent estrous synchronization with PMSG followed 48 hours later by human chorionic gonadotropin (hCG). Three weeks later, experimental mice received single intraperitoneal injections of busulfan (12 mg/kg) and cyclophosphamide (120 mg/kg), while control mice received 100 μL of vehicle (10% DMSO in PBS). After three additional weeks, experimental mice received intramuscular injections into the left thigh of either vehicle, FACS-sorted AMHR2+ mGriPSCs, or unsorted mGriPSCs. Before the stem cell injections, breast atrophy in the chemotherapy exposed mice had been noticeable (FIG. 3B), indicative of estrogen deficiency. Within 72 hours, the breast atrophy had been reversed in the mice receiving both AMHR2+ mGriPSCs, or unsorted mGriPSCs (FIG. 3B), suggesting restoration of estradiol production. Mice were followed for an additional one month. At necropsy, mice that received gonadotoxic chemotherapy exhibited greater evidence of follicular atrophy than control mice (FIG. 3C-D). While all mice receiving unsorted mGriPSCs developed large teratomas, no macroscopic or microscopic teratomas were seen in mice receiving AMHR2+ mGriPSCs (FIG. 3E-F).

### Orthotopic Intraovarian Injections of Stem Cell-Derived Ovarian Tissue Produces De Novo Generation of Functional Granulosa Cells and Mature Oocytes In Vivo.

While intramuscular injections of AMHR2+ mGriPSCs appeared to preserve hormonal function in chemotherapy treated mice, because of the absence of tumor formation, we were not technically able to retrieve the cells for subsequent analysis. Accordingly, the effect of orthotopic injection of mGriPSCs was investigated. Mice underwent estrous synchronization and then received alkylating chemotherapy. Rather than allowing the follicles to atrophy completely as in the prior experiments, we planned a shorter time interval for stem cell injections to model a rescue therapy. Therefore, the following week, mice received direct intraovarian injections of GFP-labelled unsorted or sorted AMHR2+ mGriPSCs via laparotomy. Shortening the timeframe between chemotherapy and stem cell administration also made it easier to perform surgical manipulation of the ovaries before they became too atrophic. Mice were then followed for one month. Mice then underwent a second 48-hour cycle of PMSG/hCG ovarian stimulation, followed by euthanasia.

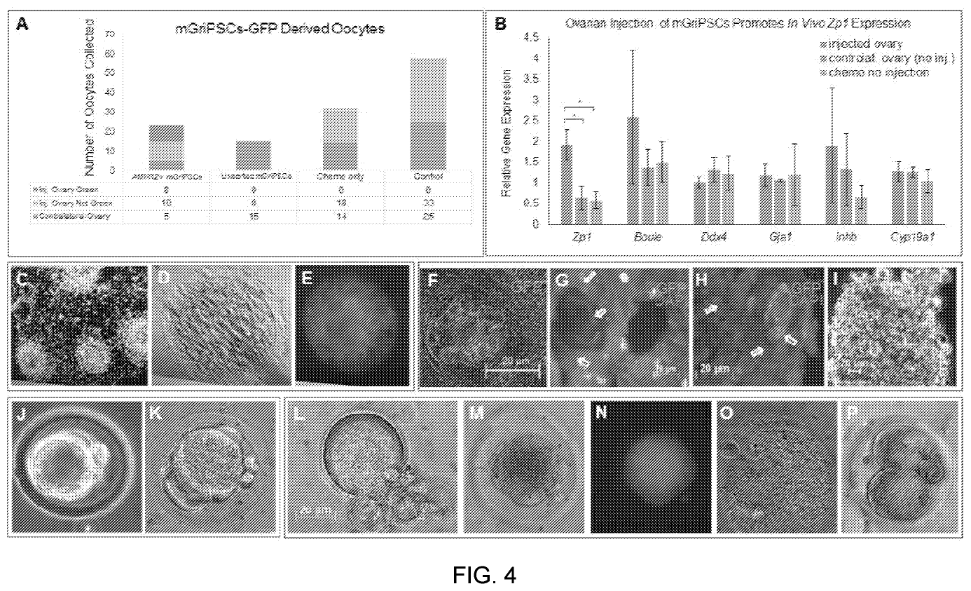

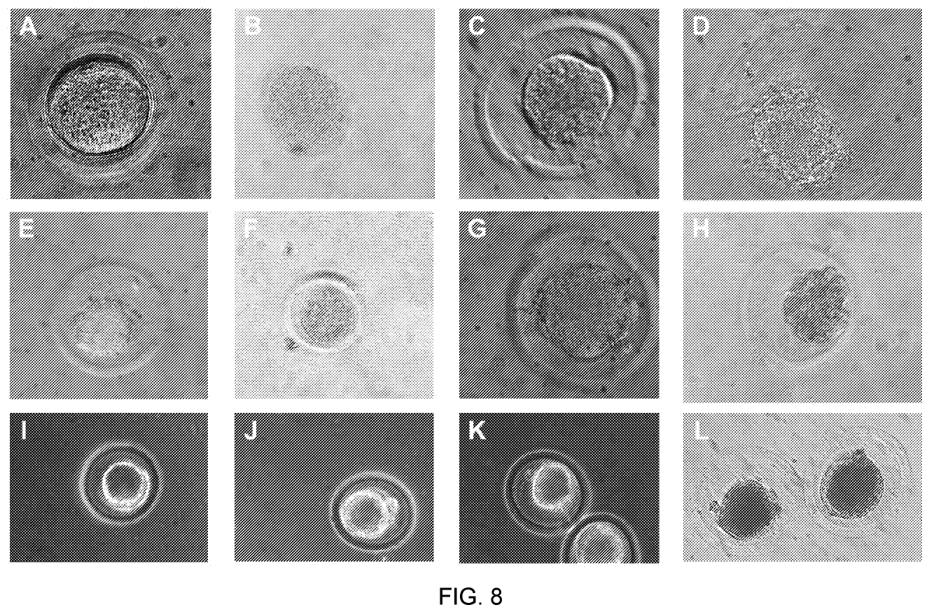

Oocytes were then collected directly from the ovaries by puncturing the bursa ex vivo and flushing the oviducts. Mature oocytes collected from AMHR2+ mGriPSC-GFP injected mice not only expressed GFP under fluorescent microscopy (FIG. 4C-E, FIG. 8A-L), but also displayed evidence of both calcium ionophore activation (FIG. 4J-K, FIG. 8I-L) and fertilization (FIG. 4L-P, FIG. 8C-H). Fertilization is further confirmed with RT-PCR analysis of co-expression of Gfp and Sty (FIG. 8M). The in-situ expression of GFP in granulosa cells of treated mice supports the de novo generation of stem cell-derived ovarian tissue, as well as oocytes (FIG. 4F, I). Ovarian follicle immunosections from AMHR2+ mGriPSC-GFP injected mice also expressed GFP, which colocalized with AMHR2 and DAZL (FIG. 4F-H). Additionally, quantitative reverse transcription PCR (RT-qPCR) indicated increased in vivo expression of oocyte marker Zp1 in the ovaries that received the sorted mGriPSCs injection compared to the contralateral, uninjected ovaries and the ovaries of mice that received chemotherapy but no stem cell injections (FIG. 4B).

18 mature oocytes were recovered from the orthotopically injected mouse ovaries after gonadotropin hyperstimulation, whereas five oocytes were collected from these mice's contralateral ovaries in which no cells were injected. Of these 18 retrieved oocytes, eight expressed GFP (FIG. 4A, FIG. 8), indicative of their stem cell origin. This indicated neogametogenesis, as well as salvage of native oocytes through paracrine effects. No oocytes were collected from the left ovaries of the unsorted injection mice, as large teratomas obstructed the collection process (FIG. 3E).

Injection of unsorted differentiated mGriPSCs into the ovary yielded the highest increase in estradiol production from pre-chemotherapy to post-chemotherapy conditions (FIG. 3G), consistent with prior observations in which heterotypic differentiated EBs synthesized high levels of steroid hormones (Anchan et al., PLOS ONE 10, e0119275 (2015); Guven et al., STEM CELLS Transl. Med. 4, 261-268(2015); Lipskind et al., Reprod. Sci. 25, 712-726 (2017); Gerami-Naini et al., Endocrinology 145, 1517-1524 (2004)). The progesterone synthesis between different experimental groups was not significantly different (FIG. 3G), as mGriPSCs appear to preferentially differentiate into estradiol synthesizing granulosa cells, presumably the consequence of their epigenetic memory. ELISA detected testosterone production in only the group that received alkylating agents without stem cell injections (FIG. 3G). Testosterone measurements support the hypothesis that observed differences between groups in estradiol levels, which is synthesized from testosterone via aromatase, are not restricted by availability of substrate. Together, this shows that AMHR2+ mGriPSCs can produce both functional granulosa cells and mature oocytes, which, according to RT-PCR, also express AMHR2 (FIG. 8).

### Stem Cell Derived Ovarian Tissue Supports Oogenesis in Co-Culture.

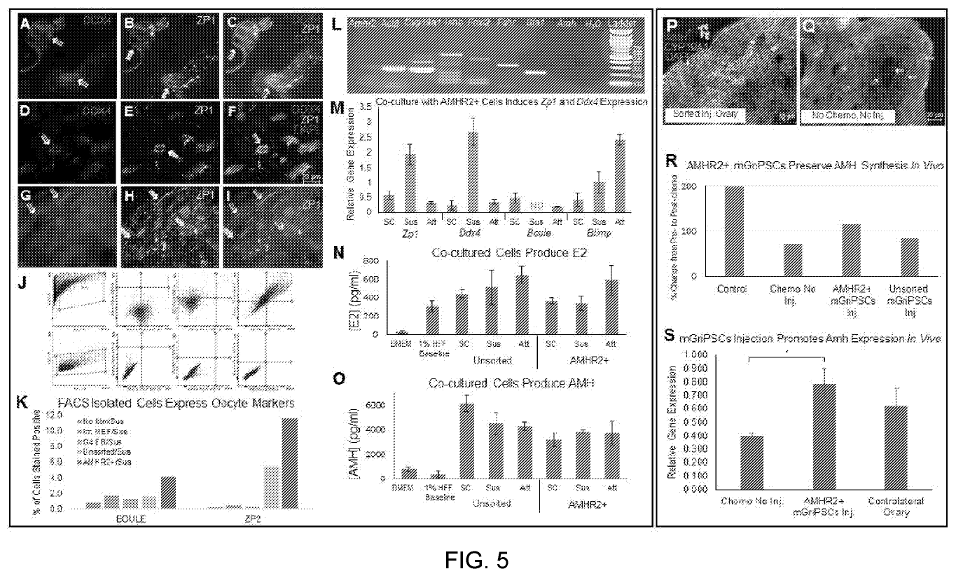

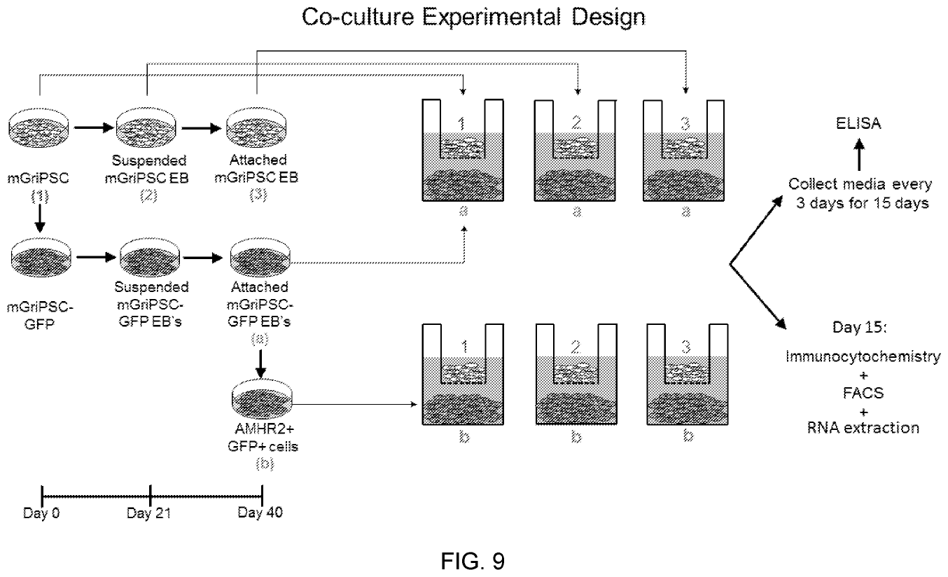

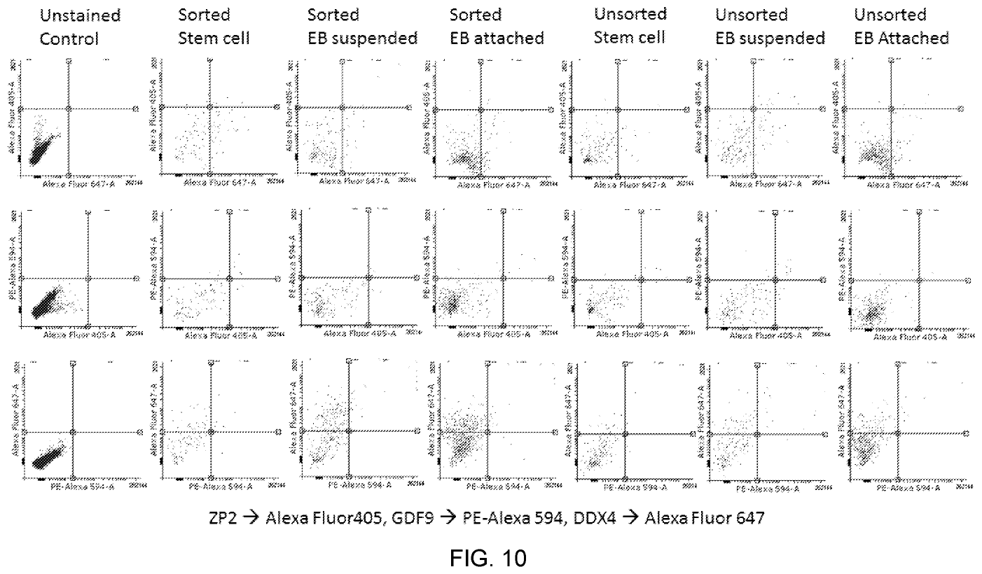

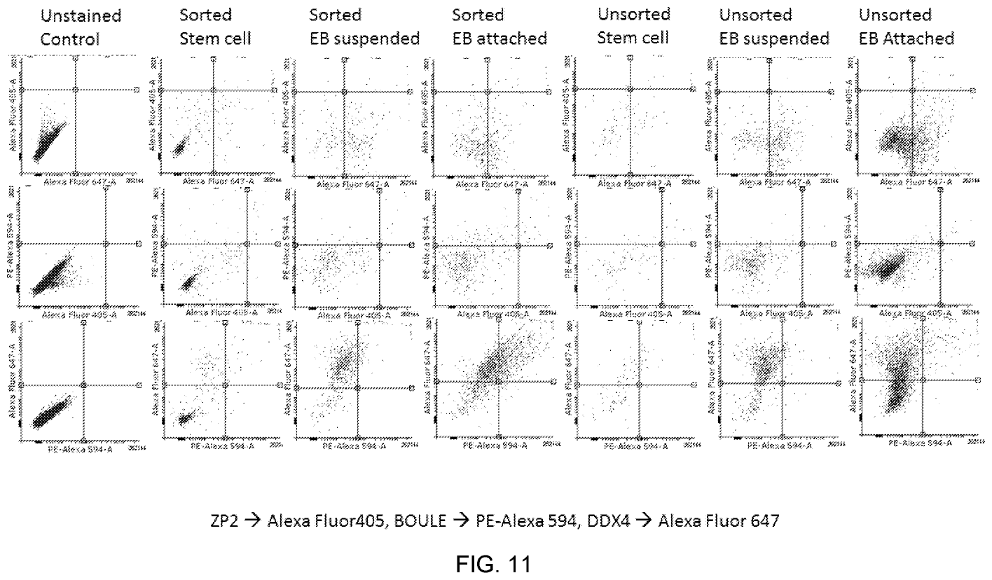

Terminal functional differentiation of de novo generated gametes from iPSCs has been attained using the described methods. We hypothesized that this is, in part, a consequence of recreating the ovarian microenvironment that supports oocyte development. Undifferentiated mGriPSCs, suspended EBs, or attached EBs were grown in transwells overlying a feeder layer of FACS-purified AMHR2+ mGriPSCs-GFP (FIG. 9). This AMHR2+ cell population reproduced the function of ovarian tissue (FIG. 9), as confirmed by transcriptome RT-PCR analysis (FIG. 5L). Under these co-culture conditions (iPSC monolayer, suspended EBs, or attached EBs), we observed the differentiation of mGriPSCs into gametes that expressed oocyte marker DDX4, as well as the terminal oocyte marker ZP1 (FIG. 5A-I). Furthermore, such differentiated cells were sorted to reveal positive expression of oocyte antigens using FACS (FIG. 5J). Compared with suspended EBs co-cultured with no feeder layer, irradiated mouse embryonic fibroblasts (MEFs), mouse G4 embryonic stem cells, or unsorted mGriPSCs, FACS showed that suspended EBs co-cultured with AMHR2+ mGriPSCs exhibited greater expression of late female oocyte markers, ZP2 and BOULE (FIG. 5J, K, FIGS. 10 and 11). Further, RT-qPCR demonstrated that suspended EBs co-cultured with AMHR2+ mGriPSCs expressed increased levels of oocyte markers ZP1 and DDX4 compared with EBs that were co-cultured with unsorted mGriPSCs (FIG. 5M). We also observed a retention of estradiol synthesis for all co-culture conditions (FIG. 5N).

### Anti-Mullerian Hormone Synthesis is Observed in Stem Cell Treated Mice.

AMH is typically observed in the native ovary, as shown by IHC of a control mouse ovary (FIG. 5Q), and is a marker for ovarian follicular reserve. The AMHR2+ FACS-purified cells that were injected into mouse ovaries demonstrated AMH synthesis in vitro (FIG. 5O) and in serum from mice treated with stem cells in vivo (FIG. 5P-S). Chemotherapy accelerated the reduction in AMH levels compared to control mice (FIG. 5R). In contrast, AMHR2+ mGriPSCs preserved AMH levels in vivo, an effect not seen with unsorted mGriPSCs (FIG. 5R). When Amh mRNA levels between the left ovary, which received AMHR2+ mGriPSCs, and the right ovary, which did not, was compared, Amh expression was high in both ovaries compared to chemotherapy exposed ovaries from mice that did not receive mGriPSCs (FIG. 5S).

These aforementioned results show that oocyte development and the supporting ovarian cortical matrix can both be promoted directly from induced pluripotent stem cells. One observation is the capacity for differentiated mGriPSs to contribute to the de novo generation of oocytes as well as form ovarian follicles as evidenced by the GFP labelled follicular granulosa cells. This observation further supports the notion that these iPSCS are regenerating ovarian tissue.

Additionally, oocyte generation from iPSCs is enhanced by using human follicular fluid, analogous to what one observes in vivo, wherein primordial germ cells are bathed in follicular fluid during critical portions of their development. The resulting oocytes generate autologous steroid and express normal phenotypic markers of germ cells. The phenotypic evidence of endocrine recovery in chemotherapy treated mice, restoration of the mammary pads, is notable as early as 72 hours after injection of the differentiated stem cells. Gonadotrophin-induced ovarian hyperstimulation results in an appropriate physiologic response by the injected cells, namely the maturation of follicles, further supporting the notion that the stem-cell derived ovarian endocrine cells exhibit normal endocrine properties as compared to that of native ovarian tissue was also observed. Additionally, AMHR2+ iPSCs was observed to facilitate resumption of oocyte development and the de novo generation of stem cell-derived oocytes, as evidenced by the presence of oocytes expressing GFP in non-GFP mice after the injection of GFP-expressing, differentiated iPSCs. Furthermore, the ability to use a calcium ionophore to activate and fertilize these stem cell-derived oocytes demonstrated truly functional neo-gametogenesis. Together, these data evidenced suggest the practicability of patients providing their own cells to promote ovarian tissue regeneration.

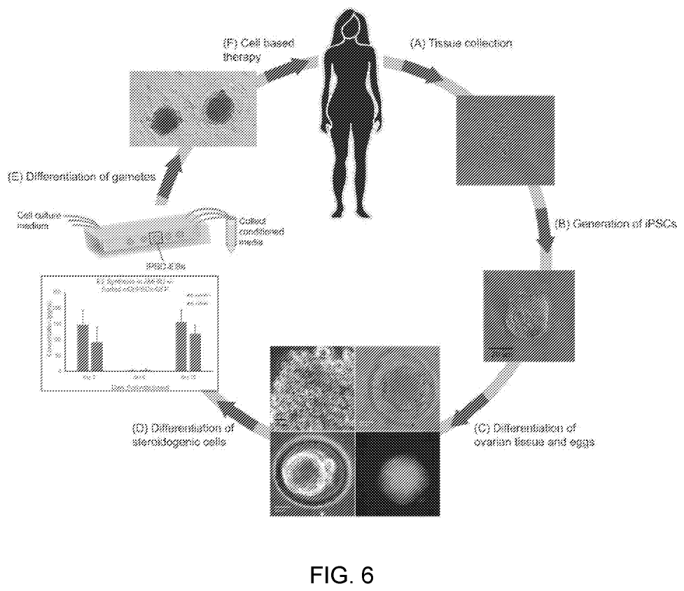

Moreover, injection of AMHR2+ mGRiPSCs into depleted ovaries of nude mice subjected to chemotherapy also improved endocrine function in the contralateral ovaries. The capacity to rescue the contralateral ovary suggests a chemoprotective effect which is presumably mediated by a paracrine mechanism. AMH synthesis in our experimental animals that received stem cell injections, observing a concurrent increase in AMH synthesis was studied. The observation of a chemoprotective effect of the contralateral ovary along with the finding of elevated AMH synthesis collectively demonstrates that AMH mediates chemoprotective effects and restoration of ovarian function. Endogenous resumption of oocyte development in stem cell treated mice is remarkable given the marked atrophy of ovarian tissue in the treated mice without injection of stem cells. Additionally, the capacity to generate physiologic concentrations of estradiol and progesterone provides patients the option of using bio identical hormones for replacement therapy (FIG. 6). By applying this to microfluidic chip technology, patient specific steroidogenic cells can be placed and cultured within a sterile, continuously flowing environment (FIG. 6). Personalized reproductive hormones (progesterone, estradiol, testosterone, and AMH) can be generated and utilized by specific extraction from conditioned media. These personalized hormones could be used for hormone replacement therapy (HRT) as well as in vitro egg maturation. These findings provide for the development of therapeutic options for cell-based therapies, especially relevant for POF patients (FIG. 6).

In sum, these results provide insight into terminal maturation of oocytes using stem cell derived ovarian cortex. The findings provide evidence for restoring both endocrine and oocyte function to ovaries damaged by chemotherapy.

### Materials and Methods

All supplies were purchased from Sigma Aldrich (St. Louis, Mo.), unless otherwise stated. All antibodies were purchased from Abcam (Cambridge, Mass.; Table 2) and all PCR primers (Table 3) were purchased from Thermofisher (Waltham, Mass.). All protocols involving animals or using animal tissue have been approved by Brigham and Women's Hospital (BWH) Institution of Animal Care and Use Committee (IUCAC), detailed in protocol #2016N000367/Dana Farber Cancer Institute (DFCI) Institution of Animal Care and Use Committee (IUCAC), detailed in protocol #15-047. All experiments were performed in accordance with relevant guidelines and regulations.

TABLE 2
Antibodies used for immunolabeling.

| Antibody | Company | Cat. # |
|---|---|---|
| AMH | Santa Cruz | Sc-6886 |
| AMHR2 | Abcam | Ab64762 |
| BOULE | Abcam | Ab104491 |
| CYP19a1 | Abcam | Ab35604 |
| DAZL | Abcam | Ab34139 |
| FSHR | Santa Cruz | Sc-7798 |
| FOXL2 | Abcam | Ab5096 |
| GJA1 | Abcam | Ab11370 |
| INH β-A | Santa Cruz | Sc-166503 |
| MVH/DDX4 | Abcam | Ab13840 |
| NANOG | Abcam | Ab106465 |
| OCT4 | Abcam | Ab18976 |
| SSEA1 | Millipore | MAB4301 |
| ZP1 | Santa Cruz | Sc-23706 |
| ZP2 | Santa Cruz | Sc-32752 |

TABLE 3
PCR Primer Sequences

| Gene | Forward Sequence | Reverse Sequence |
|---|---|---|
| Actb | TGTTACCAACTGGGACGACA | CCATCACAATGCCTGTGGTA |
| | (SEQ ID NO: 1) | (SEQ ID NO: 2) |
| Amh | GCAGTTGCTAGTCCTACATC | TCATCCGCGTGAAACAGCG |
| | (SEQ ID NO: 3) | (SEQ ID NO: 4) |
| Amhr2 | GTATCCGCTGCCTCTACAGC | AGCCTGGCTCATCACTGTCT |
| | (SEQ ID NO: 5) | (SEQ ID NO: 6) |
| Blimp1 | CTTCTCTTGGAAAAACGTGTGGG | TCATATCAGCGTCCTCCATG |
| | (SEQ ID NO: 7) | (SEQ ID NO: 8) |
| Boule | TTGTCAATCCGGCCATTTGC | CCGTATCTTGGGCCACTTGT |
| | (SEQ ID NO: 9) | (SEQ ID NO: 10) |
| Cyp19a1 | CAAAGCACGCTGCAAATACCA | GGCCAAATGTGTCTTCCAGT |
| | (SEQ ID NO: 11) | (SEQ ID NO: 12) |
| Dnmt3B | GCGCAGCGATCGGCGCCGGAGA T | CATACCCGGTGGCACCCTGTTCTTC |
| | (SEQ ID NO: 13) | AGTCA (SEQ ID NO: 14) |
| Foxl2 | GCTATTTAGGTGACACTATAGTC | TTGTAATACGACTCACTATAGGGCC |
| | ATAGCCAAGTTCCCGTTC | AGGAGTTGTTGAGGAA |
| | (SEQ ID NO: 15) | (SEQ ID NO: 16) |
| Fshr | AGTTGCATGGCATGTGTGAT | CATCACTGGGAACACCACG |
| | (SEQ ID NO: 17) | (SEQ ID NO: 18) |
| Gdf3 | ACCTTTCCAAGATGGCTCCT | CCTGAACCACAGACAGAGCA |
| | (SEQ ID NO: 19) | (SEQ ID NO: 20) |
| Gfp | CTCGCTTGTCGGCCATGATA | CACGACTTCTTCAAGTCCGC |
| | (SEQ ID NO: 21) | (SEQ ID NO: 22) |
| Gja1 | AACCTTGACTTCCGAGCAGA | CTTGGGGAACAAAGGAATCA |
| | (SEQ ID NO: 23) | (SEQ ID NO: 24) |
| Inh β-A | GCTATTTAGGTGACACTATAGA | TTGTAATACGACTCACTATAGGGCT |
| | TCATCACCTTTGCCGAGTC | TCTTCCCATCTCCATCCA |
| | (SEQ ID NO: 25) | (SEQ ID NO: 26) |
| Mvh/Dd x4 | GAGAACACATCTACAACTGGTG G | CCTCGCTTGGAAAACCCTCT |
| | (SEQ ID NO: 27) | (SEQ ID NO: 28) |
| Nanog | CAGGTGTTTGAGGGTAGCTC | CGGTTCATCATGGTACAGTC |
| | (SEQ ID NO: 29) | (SEQ ID NO: 30) |
| Oct4 | CACGAGTGGAAAGCAACTCA | CTGGGAAAGGTGTCCCTGTA |
| | (SEQ ID NO: 31) | (SEQ ID NO: 32) |
| Sry | GCTATTTAGGTGACACTATAGA | TTGTAATACGACTCACTATAGGGAA |
| | TGGAGGGCCATGTCAAG | CAGGCTGCCAATAAAAGC |
| | (SEQ ID NO: 33) | (SEQ ID NO: 34) |
| Zp1 | CCCTGAGATTGGGTCAGCG | AGAGCAGTTATTCACCTCAAACC |
| | (SEQ ID NO: 35) | (SEQ ID NO: 36) |

### Cell Lines

Mouse Granulosa Cells Retrieval

Female C57BL6/J mice were purchased from Charles River Laboratories (Wilmington, Mass.), housed at Dana Farber vivarium, and followed by a veterinarian. In accordance with IUCAC approval, female C57BL6/J mice were hyperstimulated with pregnant mare's serum gonadotropin (PMSG) and human chorionic gonadotropin (HCG). The mice were subsequently sacrificed and their oocyte-GC-complexes collected and harvested using standard techniques. Hyaluronidase was then used to release the cumulus GCs surrounding each oocyte, and the resulting GCs were centrifuged at 1500 rpm.

Generation and Expansion of mGriPSCs

Mouse granulosa cells were reprogrammed as previously described (Anchan et al., Curr. Protoc. Hum. Genet. (2017)) to generate mouse granulosa-cell derived induced pluripotent cells (mGriPSCs). Briefly, employing standard retroviral production protocols using pMXs retroviral plasmids as vectors (Kitamura et al., Exp. Hematol. 31, 1007-1014 (2003)), mouse retroviral reprogramming vectors for iPSC genes, Oct4, Sox2, c-Myc and Klf4, were created. 293T cells, in DMEM and 10% HI FBS, were cultured until 40-50% confluency, and then transfected with the reprogramming vectors stated above, ecotropic envelope (ECO) and vesicular stomatitis virus-G glycoprotein (VSV-G) using FuGENE (Roche, Indianapolis, Ind.) for 48 hours before being harvested. Due to low proliferation rates in culture, the primary GCs were infected with the viral vectors and 8 μg/ml polybrene (Millipore, Burlington, Mass.) for 24 hours and then the viral media was rinsed. Cultures were observed for 2 weeks for the presence of stem cell-like colonies, which were then morphologically determined to be picked and subcultured on mitomycin C-mitotically-inactivated mouse embryonic fibroblasts (MEFs; Global Stem, Rockville, Md.) feeder layer. Stem cells were cultured in standard mouse stem cell media for several days. Mouse stem cell media contained DMEM, 10% ES-grade HI FBS, 1000 U/ml embryonic stem cell research oversight-leukemia inhibitory factor (ESCRO LIF; Millipore), 2 mM L-Glutamine (GIBCO), 0.2 mM 2-mercaptoethanol (BME). Stem cell colonies were picked based on morphology, passaged onto fresh MEF feeder plates, and further isolated by the identification of an external antigen characteristic of undifferentiated stem cells, stage-specific embryonic antigen-1 (SSEA-1; Millipore), through live-immunostaining. The positively stained stem cell colonies were then picked and further expanded. (Results previously published Anchan et al 2017).

GFP Tagging Through Viral Infection and Stem Cell Pluripotency Verification

Infecting mGriPSCs with green fluorescent protein (GFP) would allow the mGriPSCs and resulting differentiated cells to be labeled and tracked throughout our experimental process. The GFP gene was transfected into 293T cells by combining the GFP construct, VSV-G, and delta 8.2 lentiviral packaging system with FuGENE and culture media. Using a fluorescent microscope (Nikon), the GFP signal was observed in transfected 293T cells. The viral containing culture media was harvested and mixed with 8 μg/ml polybrene to be fed onto healthy stem cell colonies. Resulting mGriPSCs expressing GFP were purified by dissociation and isolated by FACS machines. The GFP containing mGriPSCs from FACS were further purified with previous described live-staining method and stem cell colonies were verified by PCR and immunocytochemistry (ICC) as well as an alkaline-phosphatase reaction kit to ensure pluripotency. Commercial stem cell antibodies, OCT4 (Abcam), SSEA-1 (Millipore), and NANOG (Abcam), were used for ICC verification. The PCR was performed using the primers of stem cell markers Oct4, Nanog, Gdf3, and Dnmt3b to assume pluripotency of the purified GFP positive stem cell population (Anchan et al., PLOS ONE 10, e0119275 (2015)).

Embryoid Body Formation

mGriPSC colonies were manually picked based on morphology and treated with 0.05% Trypsin-EDTA (GIBCO) for 2-3 minutes to dissociate the colonies. The cells were then transferred to plates coated with 2% poly-HEMA in ethanol and cultured in suspension in EB media, consisting of DMEM-F12, 15% knock-out serum replacement (KOSR), 15% HI FBS, 1 mM L-glutamine, 0.1 mM 2-mercaptoethanol, 1% non-essential amino acids (NEAA; Invitrogen, Grand Island, N.Y.), and 1% antibiotic-antimycotic solution (Invitrogen). Cells were cultured in suspension for 20 days, changing the media to fresh EB media every three days, without disturbing the EBs. After 20 days in suspension, Ebs were transferred and attached to gelatin-coated plates for future analysis.

Fluorescence-Activated Cell Sorting

To further purify the subpopulation of presumptive ovarian and oocyte cells from the differentiated mGriPSCs-GFP cells, FACS was employed, using ovarian surface receptor, AMHR2. To prepare for FACS, Ebs were dissociated to generate single cell suspension with 0.05% trypsin EDTA and passed through a 40-μm filter. The cells were then stained with AMHR2 primary antibody for one hour, rinsed, and then treated with anti-mouse Alexa Flour 488 secondary antibody (Life Technologies/InVitrogen). Using BD FACSAria multicolor high-speed sorter and FACSDiva version 6.1.2 software (BD Biosciences, Franklin Lakes, N.J.), cells were separated into either AMHR2+ and AMHR2− groups. AMHR2+ and AMHR2− sorted cells were subsequently plated onto gelatin-coated plates and cultured in EB media for one week.

FACS was additionally utilized for cell count analysis for cells positively expressing oocyte and ovarian markers ZP1 and GJA1 in cultures containing different concentrations of HFF. mGriPSCs were cultured in EB media containing different concentrations of HFF to determine if HFF could influence the differentiation of mGriPSCs into presumptive ovarian or oocyte cells. The difference in the percentage of ZP1 and GJA1 expression from media containing 0%, 1%, and 5% HFF was calculated by FACS cell count analysis at day 9 and day 15.

FACS cell count analysis was also used to measure the expression of oocyte markers BOULE, DDX4, GDF9, and ZP2 at day 15 in each of the top layer cells from the 6 different co-culture conditions (FIGS. 10 and 11).

### Human Follicular Fluid (HFF) Acquisition

The following protocol involving human participants has been approved by Partners Human Research Committee (PHRC), the Institutional Review Board (IRB) of Partners HealthCare, protocol #2011P000795. All experiments were performed in accordance with relevant guidelines and regulations. We confirm that written informed consent was obtained from all participants. HFF was obtained from our institution's In Vitro Fertilization (IVF) laboratory as discarded tissue from consenting patients. Oocyte retrievals were performed as part of the patients' routine care. After transvaginal ultrasound-guided aspiration of oocytes as well as the follicular fluid was completed, discarded HFF was collected as part of the approved IRB protocol, anonymized and transferred to the research laboratory. Freshly obtained HFF were centrifuged at 1500 RPM for 5 min to create a pellet of cumulus granulosa cells, excessive red blood cells, and all suspended biomaterials. The supernatant was collected and passed through a 0.22 um filter to remove impurities and contaminants. Filtered HFF were frozen at −80° C. until necessary cell culture use.

### In Vitro and In Vivo Ovarian Function Restoration Experiments

In Vitro: Co-Culture Experiment

mGriPSCs were collected from various stages of their maturation, as was previously described above. Attached mGriPSC-GFP EBs or AMHR2+-GFP EBs were plated as bottom layers on a trans-well, while mGriP stem cells, suspended mGriPSCs-EBs, or attached mGriPSCs-EBs were cultured on top of the trans-wells. The different bottom and top layer cell types therefore created 6 different culture conditions. Cell cultures were grown for 15 days, collecting media every 3 days and replacing the volume of the collected media with fresh culture media (FIG. 9). This media was used for ELISAs to measure AMH and E2 concentrations at each timepoint (day 0, 3, 6, 9, 12, and 15). At day 15, the bottom cells (AMHR2+-GFP EBs) that were cultured beneath mGriPSCs were used for RNA extraction for PCR. All top layer cell types were analyzed by measuring relative gene expression of Zp1, Ddx4, Boule, and Blimp using qPCR, as well as isolating cells positively expressing BOULE, DDX4, GDF9, and ZP2 via FACS (FIGS. 10 and 11). Lastly, top layer cells were stained for DDX4 and ZP1 for immunocytochemistry analysis. Detailed experimental protocols are described below under the section titled analysis of mGriPSC and mGriPSC-GFP differentiation.

In Vivo: Ovarian Function Restoration in Chemotherapy Treated Nude Mice Mouse Gonadotoxic Treatment and Cell Injection

Female B6.Cg-Foxn1<nu>/J mice were received (Day 0) from Jackson Labs (Bar Harbor, Me.). Twelve days after arrival, eight-week old mice were superovulated with 5 IU of pregnant mare serum gonadotropin and with 5 IU of human chorionic gonadotropin after 36 more hours to synchronize the mice's menstrual cycles. On day 15, premature ovarian insufficiency was induced by single intraperitoneal injections of busulfan (12 mg/kg) and cyclophosphamide (120 mg/kg). Control mice received 100 ul of vehicle (10% DMSO in PBS). Hormone synthesis was analyzed and compared between mice that received chemotherapy and controls. The teratoma formation experiments had four mice in each control group (no treatment or chemotherapy alone) and three mice in each experimental group. For the intraovarian injections, mice in each cage were randomized to group assignments. On day 20, for each cage of five mice, one mouse received chemotherapy without stem cell rescue, and the remaining four mice underwent laparotomy with intraovarian injection of either sorted or unsorted mGriPSCs. The laparotomy was performed by placing the mice under isoflurane anesthesia, then infiltrating the ventral midline with a 1:1 mixture of lidocaine and bupicaine. A midline anterior vertical laparotomy was made under sterile technique. In each case, the left ovary was gently elevated and injected with 50 ul of cells suspended in PBS using a 27-gauge needle. The ovary was then returned to the abdomen and the abdominal wall closed in two layers using suture. Mice receive 72 hours of meloxicam for post-operative analgesia. Four cages of biologic replicates were performed. A separate cage of five control mice received vehicle injections of 10% DMS in PBS.

Oocyte Retrieval

After the mice were sacrificed, their oviducts were obtained using standard techniques. The oviducts were punctured with a 28-gauge needle and washed with 1 ml of PBS with calcium and magnesium to release the cumulus-oocyte complexes from the ampullary regions. To release the cumulus cells from surrounding the oocytes, the cumulus-oocyte complexes were treated with potassium simplex optimized medium (KSOM) with penicillin-streptomycin containing 200 IU/ml hyaluronidase at 37° C. for 3 minutes and then washed three times using fresh KSOM. Oocytes were cultured in KSOM at 37° C. and 5% CO₂.

Oocyte Activation

Oocytes were activated via calcium ionophore activation or sperm fertilization. Oocytes activated by a calcium ionophore were treating hyaluronic acid stripped oocytes with KSOM and A23187 Calcium Ionophore overnight at 37° C. in 5% CO₂. Using a phase-contrast light microscope (Zeiss, Oberkochen, Germany), oocytes, cultured in KSOM at 37° C. in 5% CO₂, were observed for 3 days for any signs of activation. Oocytes that were not activated by a calcium ionophore, were treated with sperm and observed for any signs of fertilization. To store for future RNA extraction procedures, oocytes, granulosa cells, and half of each ovary were snap frozen in liquid nitrogen. The other half of the ovary was submerged in optimal cutting temperature (OCT) compound (Thermofisher, Houston, Tex.), placed on dry ice to freeze, and stored in −80° C. freezer for future IHC analysis.

Processing of Control Ovarian Tissues

Untreated C57BL6/J mice were sacrificed, and their ovaries were excised, fixed in cold 4% paraformaldehyde/4% sucrose, and processed for paraffin embedding. Serial sectioned slides of the ovaries were stained by hematoxylin and eosin (H&E) as well as immunostained for oocyte and ovarian markers listed above. The serial sectioning of control ovaries was necessary to provide a comparative staining for our experimental cells and tissues, displaying ovarian and oocyte markers.

Analysis of mGriPSC and mGriPSC-GFP Differentiation

Ovarian and oocyte markers were used to qualitatively characterize the differentiation of mGriPSCs into presumptive ovarian and oocyte cells.

RT-PCR Analysis

RT-PCRs were performed on mGriPSCs as well as the mGriPSCs-GFP to ensure that the differentiation of the cells was not influenced by the insertion of GFP. The mGriPSCs-GFP were further analyzed by performing PCRs for both the cells before FACS and after FACS (AMHR2+ cells). PCRs were also performed for the bottom layer of co-culture cells described below using ovarian markers Cyp19a1, inhibin β-A (Inhb), forkhead box protein L2 (Foxl2), Fshr, Gja1, and Amh. After RNA was extracted using commercially available kits (Qiagen, Germantown, Md.), reverse transcribed cDNA was synthesized via a qScript cDNA Synthesis kit (Quanta Biosciences, Gaithersburg, Md.). The cDNA along with DNA polymerase (Promega, Madison, Wis.) were combined with corresponding primers for ovarian markers (anti-Müllerian hormone receptor (Amhr2), aromatase (Cyp19a1), follicle-stimulating hormone receptor (Fshr), gap junction alpha-1 (Gja1), and anti-Müllerian hormone (Amh)) and oocyte markers (DEAD-box helicase 4 (Ddx4), Boule, and PR domain zinc finger protein-1 (Blimp-1)), with β-actin as a positive control to analyze the differentiation of mGriPSCs. Cycling conditions were 95° C. for 3 minutes, 35 repetitions of (95° C. for 1 minute, 58.5° C. for 1 minute, and 72 for 1 minute) and 72° C. for 10 minutes in a thermocycler (Bio-Rad). Amplified products were separated on 1.0% agarose gel electrophoresis (Thermofisher) to qualitatively analyze the expression of tested biomarkers.

qPCR Analysis

Although the commercially available RNA extraction and cDNA synthesis kits described above were used for mGriPSCs, freshly harvested tissue from 3 sacrificed mice in each condition were snap-frozen in liquid nitrogen for 30 seconds and stored in −80° C. overnight. Frozen samples were then thawed and ground with mortar and pestle until homogenized with lysate buffer.

These homogenized and lysed tissue samples were then processed for RNA extraction as described above. After cDNA synthesis, 2 ng of cDNA was used in each qPCR reaction well. Primers for the oocyte markers listed above with the addition of oocyte marker Zona Pellucida-1 (Zp1) were used for qPCR analysis of co-cultured cells, while Zp1, Boule, Ddx4, Gja1, Inhb, Cyp19a1, and Amh were used for primers for qPCR of ovary tissues. Each set of primers as well as Power SYBR Green reaction mix (Applied Biosystems, Foster City, Calif.) were used for qPCR reactions. The QuantStudio 3 (Applied Biosystems) qPCR machine was used with the cycling conditions of 50° C. for 2 minutes, 95° C. for 10 minutes, and 60 repetitions of (95° C. for 1 minute and 60° C. for 2.5 minutes). Double-delta CT analysis was used with β-actin as the housekeeping gene. qPCRs for the ovarian tissue used ovaries that were not treated with chemotherapy nor injected with stem cells as the control from which fold change in gene expression was measured in the treated ovaries. mGriPSCs-GFP in one of three developmental stages (1. Stem cells, 2. Suspended EBs, or 3. Attached EBs) were co-cultured on the top layer of a transwell with either sorted AMHR2+ mGriPSCs-GFP or unsorted, differentiated mGriPSCs-GFP on the bottom layer. qPCR was used to quantify relative gene expression of oocyte markers in the top layer of cells, measuring the effect of co-culturing with sorted versus unsorted cells on the bottom layer. When analyzing relative gene expression via delta-delta CT, cells from each developmental stage were compared only with cells from the same developmental stage, thus focusing the analysis on the effect of co-culturing with sorted cells vs unsorted cells.

Immunocytochemistry/Immunohistochemistry (ICC/IHC)

Pre-warmed 0.05% Trypsin-EDTA was used to dissociate mGriPSC EBs so that they could be subsequently reattached to gelatin-coated plates as a more ideal visual monolayer of cells. Cells or prepared histological tissue samples were fixed in cold 4% paraformaldehyde (4° C.)/4% sucrose for 30 minutes at room temperature and rinsed three times with 1× phosphate-buffered saline (PBS; Corning, Corning, N.Y.) for 5 minutes. The cells were blocked with 2% donkey serum, 10 mg/mL bovine serum albumin, and 1% Triton-X for 30 minutes. The primary antibodies for ovarian markers, AMHR2, CYP19a1, FOXL2, FSHR (Santa Cruz, Dallas, Tex.), INHB (Santa Cruz), GJA1, and primary antibodies for oocyte markers, Deleted in zoospermia-like (DAZL), DEAD-Box Helicase 4 (DDX4), zona pellucida glycoprotein 1 (ZP1; Santa Cruz), and zona pellucida glycoprotein 2 (ZP2; Santa Cruz), were then applied for 2 hours at room temperature, followed by three 5 min PBS rinses before the secondary antibodies (Thermofisher) were applied for 1 hour in a dark environment at room temperature. After the secondary antibody incubation, the cell and tissue samples were rinsed 3 times for 5 minutes with PBS, followed by the application of 4′,6-diamidino-2-phenylindole (DAPI) for 10 minutes to visualize the nuclei. Finally, the samples were rinsed three last times with PBS for 5 minutes before performing fluorescent microscopy using a Zeiss Axiovert (Zeiss Microscopes) 40 CFL and ImagerM2.

ELISA and Hormone Analysis

Estradiol concentrations within attached EB media were analyzed using commercially available enzyme-linked immunosorbent assay (ELISA) kits (Abnova, Zhongli, Taiwan). Three media samples from each condition were collected at day 0, 6, 9, 12, and 15. Additionally, estradiol concentrations from media containing 0%, 0.5%, 1%, 2%, 3%, and 5% HFF were measured at day 0, 9, and 15 using ELISA kits. Estradiol, progesterone, and Anti Mullerian hormone (AMH) concentrations were also analyzed via ELISA kits for either the mGriPSC-GFP pre-FACS and post-FACS cell analysis at days 0, 3, 6, 9, 12, and 15, or for the 6 different in vitro co-culture conditions at days 0, 3, 6, 9, 12, and 15, or from blood serum from the four different mouse injection groups at three timepoints (baseline, pre-chemo, and pre-sacrifice) from the in vivo mouse injections.

### Other Embodiments

Other embodiments are within the following claims.

What is claimed is:

1. A container comprising (i) a substrate; (ii) human iPSCs capable of differentiating into functional ovarian tissue; and (ii) a culture media.

2. The container of claim 1, wherein the container is configured such that the culture media continuously flows through the container.

3. The container of claim 2, the container having an inlet port and an outlet port configured such that media flows through the container.

4. The container of claim 1, wherein the iPSCs are matched to an individual female.

5. The container of claim 1, wherein the substrate is a microfluidic chip.

6. The container of claim 1, further comprising human embryoid bodies.

7. The container of claim 1, further comprising human steroidogenic cells.

8. The container of claim 1, further comprising human ovarian tissue.

9. The container of claim 1, further comprising human oocytes.

10. The container of claim 1, further comprising human reproductive hormones.

11. The container of claim 10, wherein the hormones are progesterone, estradiol, testosterone, or anti-Müllerian hormone (AMH) or a combination thereof.

12. The container of claim 1, wherein the culture medium comprises human follicular fluid (HFF).

13. Ovarian tissue produced by the steps of
i) providing human iPSCs on a substrate;
ii) differentiating the iPSCs into ovarian tissue; and
iii) harvesting the ovarian tissue.

14. The ovarian tissue of claim 13, wherein the substrate is a microfluidic chip.

15. The ovarian tissue of claim 13, wherein the iPSCs are autologous.

16. Human oocytes produced by the steps of
i) providing human iPSCs on a substrate;
ii) differentiating the iPSCs into oocytes; and
iii) harvesting the oocytes.

17. The oocytes of claim 16, wherein the substrate is a microfluidic chip.

18. The oocytes of claim 16, wherein the iPSCs are autologous.

19. Conditioned media produced by the steps of
i) providing human iPSCs on a substrate;
ii) providing a cell culture medium which contacts and continuously flows over the iPSCs;
iii) differentiating the iPSCs into ovarian tissue or oocytes; and
iv) collecting, following the contacting, the media, thereby producing conditioned media.

20. The conditioned media of claim 19, wherein the substrate is a microfluidic chip.

21. The conditioned media of claim 19, wherein the iPSCs are autologous.

22. The conditioned media of claim 19, wherein the cell culture medium of ii) comprises HFF.

23. The conditioned media of claim 19, wherein the collected conditioned media comprises reproductive hormones.

24. The conditioned media of claim 23, wherein the reproductive hormones are progesterone, estradiol, testosterone, or AMH or a combination thereof.

25. A method of treating a fertility or endocrine condition in a human female, the method comprising administering the human ovarian tissue produced according to claim 16 to the female.

26. The method of claim 25, wherein administering comprises implanting.

27. The method of claim 24, further comprising administering conditioned media.

28. The method of claim 25, wherein the fertility or endocrine condition is selected from the group consisting of premature menopause, premature ovarian insufficiency, infertility, chemotherapy-induced premature ovarian failure, chemotherapy-induced diminished ovarian reserve, chemotherapy-induced decreased ovarian reserve, idiopathic premature ovarian failure, chemotherapy-induced ovarian failure, chemotherapy-induced premature ovarian insufficiency, estrogen deficiency, age-related rapid follicular atresia, follicular atrophy, post-chemotherapy ovarian insufficiency, and oophorectomy.

29. A method of treating a fertility or endocrine condition in a human female, the method comprising administering the human oocytes produced according to claim 19 to the female.

30. The method of claim 29, wherein administering comprises implanting.

31. The method of claim 29, further comprising administering conditioned media.

32. The method of claim 29, wherein the fertility or endocrine condition is premature ovarian insufficiency, infertility, chemotherapy-induced premature ovarian failure, chemotherapy-induced diminished ovarian reserve, chemotherapy-induced decreased ovarian reserve, idiopathic premature ovarian failure, chemotherapy-induced ovarian failure, chemotherapy-induced premature ovarian insufficiency, post-chemotherapy ovarian insufficiency, and oophorectomy.

33. A method of providing a hormone replacement therapy to a human female, the method comprising administering the conditioned media produced according to claim 19 to the female.

34. The method of claim 33, wherein the conditioned media comprises human reproductive hormones.

35. The method of claim 34, wherein the hormones are progesterone, estradiol, testosterone, or AMH or a combination thereof.
